(19) United States
(12) Patent Application Publication
(10) Pub. No.: US 2026/0157880 A1
(43) Pub. Date: Jun. 11, 2026

(54) DEVICE FOR AND METHOD OF NUMBING THE EYE OF A LIVING BEING

(71) Applicant: Eyedocaine, Inc., Philadelphia, PA (US)

(72) Inventor: Joshua M. Greene, Villanova, PA (US)

(73) Assignee: Eyedocaine, Inc., Philadelphia, PA (US)

(21) Appl. No.: 19/414,579

(22) Filed: Dec. 10, 2025

Related U.S. Application Data
(60) Provisional application No. 63/730,501, filed on Dec. 11, 2024.

(51) Int. Cl. A61F 9/00; A61K 31/167

(52) U.S. Cl. CPC A61F 9/0017; A61K 31/167

(57) ABSTRACT

A device and method for numbing an eye of a patient. The device includes an elongated flexible strip having a distal end portion including an absorbent patch carrying an anesthetic agent, and a proximal end portion carrying an adhesive. The absorbent patch is folded along a fold line to form a hook-like tab which is inserted into the conjunctival sac of the eye to numb it, with the proximal end portion disposed confronting the patient’s cheek, whereupon the adhesive patch is brought into engagement with the patient’s cheek to secure the device in place. The anesthetic agent may be liquid lidocaine into which the absorbent patch is soaked. The adhesive patch is protected by a removable cover.

### CROSS REFERENCE TO RELATED APPLICATION

This application claims the benefit of U.S. Provisional Patent Application No. 63/730,501, filed December 11, 2024, the disclosure of which is incorporated herein by reference in its entirety.

### FIELD

This invention relates generally to medical devices and methods and more particularly to devices and methods for numbing the eye of a living being to enable an ophthalmological procedure, e. g,. intravitreal injection, to be performed on the eye.

### BACKGROUND

Intravitreal injections are the most common ophthalmological procedure performed in an outpatient retinal setting. They are used to deliver various medicines into the vitreous space to treat complications from diabetes, hypertension, sickle cell disease, birth defects, etc. They are also used in therapeutic procedures to deliver intravitreal gas for pneumatic retinopexies: a less invasive office procedures used to spare patients from having surgery for retinal detachments. There is a myriad of medications designed for intravitreal delivery, and they are all delivered in the same way: through the conjunctiva and sclera, into the vitreous space. To perform these ophthalmologicalprocedures, physicians need to anesthetize the eye and sterilize the surface before the injection.

There is much heterogeneity in the use of anesthetics for injections. Some doctors only use topical drops, which provide some surface anesthesia, but little for ultimate penetration, and patients frequently complain of pain after this process. There is also a lidocaine gel that is used. This gel is widely used to anesthetize the eye for cataract surgery, and it has had success in the office for these injections. Some drawbacks of both drops and gel are that the technicians assisting the doctors need to go back into the examination room, over and over, to deliver the anesthetic. This pulls them away from working-up patients, which may adversely impede patient flow and impact patient volume. Moreover, long-term patients will often request a custom amount of anesthetic (e.g., number of drops), which is cumbersome and difficult for technicians to adhere to.

Another option is subconjunctival lidocaine. In this process, the patient is given a series of topical drops that numb the surface of the eye. Once numbed, the physician will inject a small amount of lidocaine under the surface layer of the eye. The lidocaine is left in place for 5-7 minutes, then the eye is cleaned and injected. This process provides excellent anesthetic, but it also may have drawbacks. It increases the chances of a subconjunctival hemorrhage, which is blood on the surface of the eye. This is unsightly and irritating. Furthermore, the blood may sometimes linger for up to a week after the injection. Patients are also unhappy about having to get two injections.

There is also the use of pledgets, i.e., a tiny cotton ball with a string attached. The cotton ball is soaked in 4% lidocaine and stuffed inside the lower eyelid so that it is held in place in between the lower eyelid and the surface of the eyeball (i.e., the conjunctival sac between the lower palpebral conjunctiva and the bulbar conjunctiva). This is effective and less invasive, however when the patients move their eyes, the cotton ball may get dislodged and fall out. If this occurs, the cotton is replaced, and the process is repeated. This process may be cumbersome for patients and technicians and takes at least 10-15 minutes depending on whether the cotton ball stays in place.

The patent literature includes some patents/applications disclosing devices for ocular delivery of medications. Examples of such patents/applications are: United States Letters Patent No. 3,416,530 (Ness) directed to an eyeball medication dispensing tablet; United States Published Application No. US 2006/0210604 A1 directed to ocular delivery of polymeric delivery formulations; and Chinese Application No. CN101437478A directed to ocular delivery of polymeric delivery formulations.

While the above-described prior art devices and/or methods may be viable for their intended purposes they nevertheless leave much to be desired from the standpoints of simplicity of construction, low-cost, ease of use, efficiency, and effectiveness.

Thus, a need exists for a device and method of use which addresses those drawbacks of the prior art. The subject invention does so by providing a device and method of use for readily numbing the eye of a living being.

### SUMMARY

One aspect of this invention is a device for numbing the eye of a living being having a globe, a conjunctival sac, a lower palpebral conjunctiva, and a bulbar conjunctiva. The device comprises a strip of flexible material, a foldable line, an absorbent patch, and an adhesive patch. The strip of flexible material has a distal end portion, a proximal end portion, and an intermediate portion located between the distal end portion and the proximal end portion. The foldable line forms an interface between the distal end portion and the intermediate portion. The absorbent patch is on the distal end portion and carries an anesthetic agent. The distal end portion is configured to be folded along the foldable line to form a hook-like tab. The hook-like tab is configured to be introduced into the conjunctival sac between the lower palpebral conjunctiva and the bulbar conjunctiva, whereupon the anesthetic agent engages a portion of the bulbar conjunctiva to numb the eye with the intermediate portion and the proximal end portion of the strip hanging down from the eye and over an external portion of the anatomy of the living being below the eye. The adhesive patch is located on the proximal end portion and is configured to be brought into engagement with the external portion of the anatomy of the living being below the eye to hold the device in place.

In accordance with one preferred aspect of the device of this invention the strip of flexible material may have an outer surface, and an inner surface. The distal end portion terminates at a distal edge, and the proximal end portion terminates at a proximal edge. The absorbent patch is on the outer surface of the distal end portion. The adhesive patch is located on the inner surface of the proximal end portion adjacent to the proximal edge.

In accordance with another preferred aspect of the device of this invention the strip of flexible material may have a length in the range of approximately 90 millimeters (mm) to 100 mm between the distal edge and the proximal edge, a width in the range of approximately 10 mm to 15 mm, and a thickness in the range of approximately 0.5 mm to 1 mm.

In accordance with another preferred aspect of the device of this invention the strip of flexible material is approximately 100 mm (about 3. 94 inch) long, approximately 10 mm wide, and approximately 0.5 mm (about 0.02 inch) thick.

In accordance with another preferred aspect of the device of this invention the hook-like tab may have a length in the range of 7 mm to 9 mm. In accordance with another preferred aspect of the device of this invention the hook-like tab may have a length of approximately 10 mm (about 0.39 inch).

In accordance with another preferred aspect of the device of this invention the flexible strip is formed of cardboard or paper.

In accordance with another preferred aspect of the device of this invention the absorbent patch may have a thickness in the range of approximately 0.5 mm to 1 mm.

In accordance with another preferred aspect of the device of this invention the absorbent patch may have a thickness of approximately 0.5 mm.

In accordance with another preferred aspect of the device of this invention the absorbent patch comprises gauze or foam.

In accordance with another preferred aspect of the device of this invention the adhesive patch may have a length in the range of 15 mm to 20 mm.

In accordance with another preferred aspect of the device of this invention the adhesive patch may have a length of approximately 15 mm.

In accordance with another preferred aspect of the device of this invention the adhesive patch is a double-sided tap and the device additionally comprises a removable cover disposed over the adhesive patch to protect the adhesive patch until the device is ready for use, whereupon the removable cover may be grasped to remove it from the adhesive patch to expose the adhesive patch.

In accordance with another preferred aspect of the device of this invention the anesthetic agent comprises lidocaine.

Another aspect of this invention is a method of numbing an eye of a living being having a globe, a conjunctival sac, a lower palpebral conjunctiva, and a bulbar conjunctiva. the method comprising providing a device comprising a strip of flexible material having a distal end portion, a proximal end portion, an intermediate portion located between the distal end portion and the proximal end portion, a foldable line forming an interface between the distal end portion and the intermediate portion, an absorbent patch carrying an anesthetic agent located on the distal end portion, and an adhesive patch located on the proximal end portion. The distal end portion is folded along the foldable line to form a hook-like tab. The hook-like tab is introduced into the conjunctival sac between the lower palpebral conjunctiva and the bulbar conjunctiva, whereupon the anesthetic agent engages a portion of the bulbar conjunctiva to numb the eye with the intermediate portion and the proximal end portion of the strip hanging down from the eye and over an external portion of the anatomy of the living being below the eye. The adhesive patch is brought into engagement with the external portion of the anatomy of the living below the eye to hold the device in place.

In accordance with one preferred aspect of the method of this invention the absorbent patch of the device is initially provided without the anesthetic agent. The anesthetic agent may be provided as a liquid and applied to the absorbent patch to be absorbed thereby. The absorbed anesthetic agent is thereafter carried by the absorbent patch. In an embodiment, the anesthetic agent may be provided as a gel and applied to the absorbent patch. The anesthetic agent gel is again, thereafter, carried by the absorbent patch.

In accordance with another preferred aspect of the method of this invention the adhesive patch is a double-sided tape, and the device additionally comprises a removable cover disposed over the adhesive patch to protect the adhesive patch until the device is ready for use. The method additionally comprises grasping the removable cover to remove it from the adhesive patch to expose the adhesive patch and thereafter bringing the adhesive patch into engagement with the external portion of the anatomy of the living below the eye to hold the device in place.

In accordance with another preferred aspect of the method of this invention the method additionally comprising leaving the device in place on the living being for a sufficient time for the eye to be numb and once numb cleaning the eye and then preforming a further procedure thereon.

In accordance with another preferred aspect of the method of this invention the anesthetic agent comprises 4% lidocaine, and wherein the sufficient time is approximately 5 to 7 minutes.

In accordance with another preferred aspect of the method of this invention the procedure comprises injecting the eye.

### BRIEF DESCRIPTION OF THE DRAWINGS

FIG. 1 is an illustration of the face of a living being, e.g., a person, on which the device of this invention has been placed to numb that being’s eye.

FIG. 2 is a side elevation view of the device shown in FIG. 1.

FIG. 3 is an isometric view of the device of FIGS. 1 and 2 before application to the living being.

FIG. 4 is an enlarged cross-sectional view of the eye of a living being, e.g., a person, with the eye of the person shown ready for receipt of the device of FIGS. 1 - 3.

FIG. 5 is an enlarged cross-sectional view of the eye of a living being, e.g., a person, like that shown in FIG. 4, but with the device of this invention in place numbing the eye.

### DETAILED DESCRIPTION

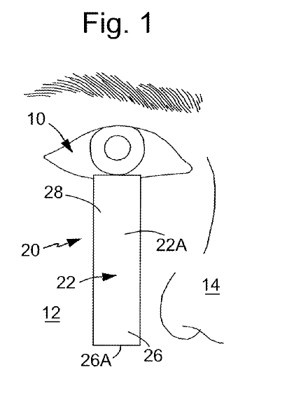

Referring now to the various figures of the drawing wherein like reference characters refer to like parts, there is shown in FIG. 1 one exemplary embodiment of a device constructed in accordance with one exemplary preferred embodiment of this invention for numbing the eye of a living being.

One example embodiment of this invention will now be described more fully with reference to the accompanying drawings. Mention of other potential exemplary embodiments may also be provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that an example embodiment may be embodied in many different forms and that neither should be construed to limit the scope of the disclosure. In some example embodiments, well-known processes, well-known device structures, and well-known technologies may not be described in detail.

The terminology used herein is for the purpose of describing particular exemplary embodiments only and is not intended to be limiting. As used herein, the singular forms ''a," ''an," and ''the'' may be intended to include the plural forms as well, unless the context clearly indicates otherwise. The terms ''comprises,” "comprising," ''including," and ''having," are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the order discussed or illustrated, unless specifically identified as an order of performance. It is also to be understood that additional or alternative steps may be taken.

When an element or layer is referred to as being ''on," ''engaged to," ''connected to," or ''coupled to'' another element or layer, it may be directly on, engaged, connected or coupled to the other element or layer, or intervening elements or layers may be present. In contrast, when an element is referred to as being “directly on," ''directly engaged to," ''directly connected to," or ''directly coupled to'' another element or layer, there may be no intervening elements or layers present. Other words used to describe the relationship between elements should be interpreted in a like fashion (e.g., ''between'' versus ''directly between," ''adjacent'' versus ''directly adjacent," etc. ). As used herein, the term ''and/or'' includes any and all combinations of one or more of the associated listed items.

Although terms such as first, second, third, etc. may be used herein to describe various elements, components, regions, layers and/or sections, these elements, components, regions, layers, and/or sections should not be limited by these terms. These terms may be only used to distinguish one element, component, region, layer or section from another region, layer or section. Terms such as ''first," ''second," and other numerical terms if used herein do not imply a sequence or order unless clearly indicated by the context. Thus, a first element, component, region, layer or section discussed below could be termed a second element, com ponent, region, layer or section without departing from the teachings of the example embodiments.

Spatially relative terms, such as ''inner," ''outer," ''beneath '' ''below'' ''lower'' ''above'' ''upper'' “front” “rear” “top” “bottom” and the like may be used herein for ease of description to' describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as ''below'' or ''beneath'' other elements or features would then be oriented ''above'' the other elements or features. Thus, the example term ''below'' may encompass both an orientation of above and below. The device may be otherwise oriented (rotated 90 degrees or at other orientations) and the spatially relative descriptors used herein interpreted accordingly.

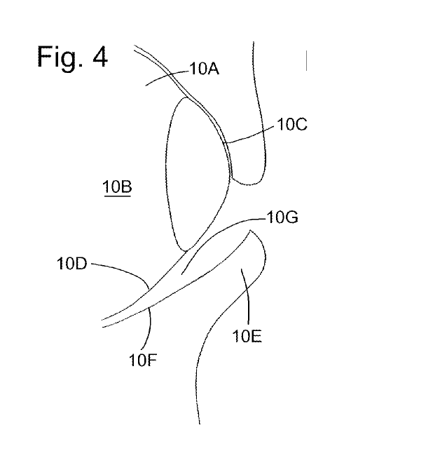

In FIG. 1 there is shown one exemplary embodiment of a device 20 constructed in accordance with the subject invention shown in place with respect to the eye 10 of a living being, e.g., a human patient. In that figure the device 20 is shown with portion of it located within the conjunctival sac of the eye, and with another portion of the device shown releasably secured to the patient’s cheek portion 12 located below the eye and beside the patient’s nose 14. Before describing the details of device 20 a brief description of the anatomy of the patient relevant to this invention will now be made with reference to FIG. 4. That figure shows the eye 10 with the lower eyelid 10E pulled back so that the eye is ready to receive device 20 of the subject invention. As may be seen that the eye 10 of patient 12 comprises the eyeball (also commonly referred to as the “globe”) 10A. Among other things the eyeball or globe includes are the vitreous body 10B, the cornea 10C, and the bulbar conjunctiva 10D which is located below the cornea. The lower eyelid 10E includes the lower palpebral conjunctiva 10F. As mentioned above, in FIG. 4 the lower eyelid 10E is shown pulled back to open the conjunctival sac 10G, i.e., the space between the lower palpebral conjunctiva 10F and the bulbar conjunctiva 10D. It is into the conjunctival sac 10G that a portion of device 20 will be introduced.

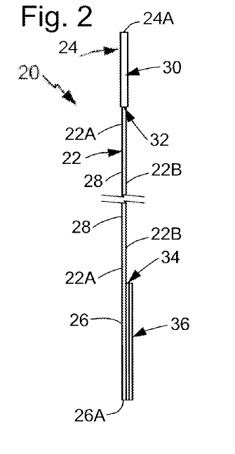

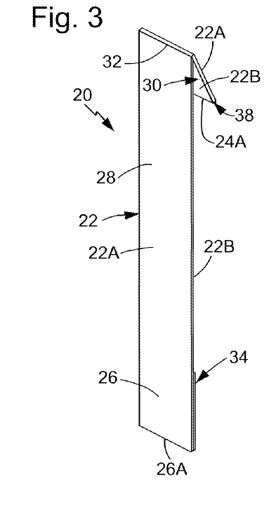

The device 20 of this invention is best seen in FIGS. 2 and 3 and basically comprises an elongated narrow flexible strip 22. The strip 22 includes a distal end portion 24, a proximal end portion 26, and an intermediate portion 28 between the distal end portion 24 and the proximal end portion 26. The distal end portion terminates at a distal edge 24A. The proximal end portion 26 terminates at a proximal edge 26A. Strip 22 includes an outer surface 22A and an inner surface 22B. The strip 22 may be made of any suitable biocompatible material. One particularly suitable and inexpensive material is cardboard or paper. The strip 22 is sufficiently sized for use with a particular patient. Thus, for example for human adult patients the strip 22 may have a length measured between the distal edge 24A and the proximal edge 26A in the range of approximately 90 mm to 100 mm, and preferably approximately 100 mm. The strip 22 may have a width measured between its lateral side edges in the range of approximately 10 mm to 15 mm, and preferably 10 mm. The strip 22 may have a thickness in the range of approximately 0.5 mm to 1 mm, and preferably 0.5 mm.

As shown, the strip 22 includes an absorbent patch 30, a foldable line 32, an adhesive patch 34, and a cover 36. The absorbent patch 30 is located on the distal end portion 24 of the strip 22 contiguous with the distal edge 24A, The absorbent patch 30 carries an anesthetic agent, e.g., 4% lidocaine, and is preferably formed of a thin layer of gauze or foam which is wrapped about the distal end portion 24 of the strip 22 so that the lidocaine held within the absorbent patch 30 will be exposed from both the inner surface of the strip 22 and the outer surface of the strip 22 to effect the numbing of the eye as will be described later.

The foldable line 32 is at the interface of the distal end portion 24 and the intermediate portion 28 of the strip 22. The foldable line enables the distal end portion 24 to be folded along the line so that the distal end portion 24 contiguous with the distal edge 24A forms a hook-like tab 38 like shown in FIG. 3.

The adhesive patch 34 is located and secured on the inner surface 22B of the proximal end portion 26 of the strip 22 contiguous with the proximal edge 26A. It is formed of any suitable, biocompatible adhesive. In the exemplary embodiment shown the adhesive patch 34 is formed of double-sided tape. To protect the exposed surface of the double-sided tape, a releasably securable cover 36, formed of any suitable material (e.g., a flexible plastic) is disposed on and releasably secured to the adhesive patch 34. The cover is arranged so that it may be grasped and removed, e.g., peeled off the double-sided tape, to expose the adhesive of the tape when device 20 is to be secured in place, as will be described later.

Use of device 20 is as follows. The device 20, if disposed in some carrier container or package, is removed. The absorbent patch 30 at the distal end portion 24 of the device may then be soaked in a liquid 4% lidocaine so that the material making up the patch 30 absorbs the 4% lidocaine. In another form, the anesthetic agent may be provided as a gel to the absorbent patch 30. The gel may also be a lidocaine material or similar anesthetic solution. The anesthetic agent gel is carried by the absorbent patch 30.

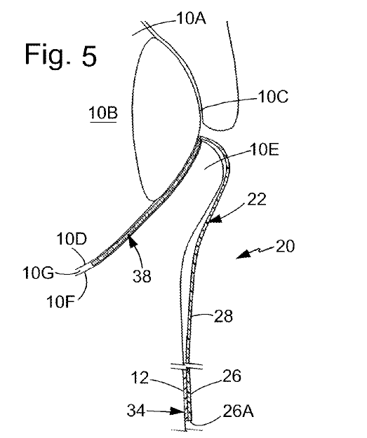

That distal end portion 24 of the strip 22 may then be folded along the foldable line 32 to form the hook-like tab 38. The cover 36 may then be removed from the adhesive patch 34. The device is now ready for application to the patient, whereupon the hook-like tab 38 carrying the anesthetic agent may be placed in the conjunctival sac 10G. To do that, the lower eyelid 10E is pulled back to the position shown in FIG. 4, and the distal edge 24A of the hook-like tab 38 is introduced over the lower eyelid 10E and into the conjunctival sac 10G. Once the hook-like tab 38 is located within the conjunctival sac 10G, the lower eyelid 10E may be released. As shown in FIG. 5, the hook-like tab 38 trapped the conjunctival sac 10G. The remaining portion of device 20 will drop down the face of the patient. The strip 20 is disposed over the patient’s face below the eye and the adhesive patch 34 is positioned adjacent the patient’s cheek 12. The outer surface 22A of the proximal end portion 26 of the strip 22 may then be pressed to cause the adhesive patch 34 to engage the patient’s cheek 12. That action releasably secures the patch 34 and the proximal end 26 of the strip 22 in place on the patient.

When the device 20 is in place as just described the outer surface 22A of the tab 38 contiguous with the distal edge 24A will carry the anesthetic agent as will the inner surface 22B of that tab. Accordingly, when that device 20 is releasably secured in place on the patient, like shown in FIG. 5, the anesthetic agent on the outer surface of the strip 22 is in engagement with the bulbar conjunctiva 10D and the anesthetic agent on the inner surface of the strip 22 is in engagement with the palpebral conjunctiva 10F. The device may then be left in place for a suitable period to numb the eye, e.g., 5 to 7 minutes using 4% lidocaine. Once the eye is sufficient numb the device may be removed by grasping the proximal end portion of the strip 22 to peel it off the patient’s cheek 12. Once the distal end portion of the device is free of the patient’s cheek 12, it may be pulled so that the hook-like tab exits the conjunctival sac and is out of the eye. The eye may then be cleaned, and then whatever procedure is to be carried out on the numbed eye may be accomplished, e.g., intravitreal delivery of a drug via injection into the vitreal space 10B.

As should be appreciated by those skilled in the art, device 20 of this invention and its method of use (e.g., the strip soaked, hooked, and fastened in place) is easy for a technician to accomplish and provides a broad area of tissue anesthetized. Moreover, the subject invention provides a relatively straightforward way to achieve consistent anesthesia that physicians, patients, and technicians may count on.

It should be noted that the subject invention, which is shown as being used for numbing the eye of a person, is not limited to only humans, but may be used in veterinary medicine for numbing the eye of an animal. While the device of this invention has been described as formed of a strip of cardboard or paper, other flexible, biocompatible materials, e.g., plastics, etc., may be used for the strip, and may be of a size sufficient for the anatomy of the living being. Furthermore, the anesthetic agent need not be lidocaine but may be any desirable agent suitable for numbing the eye. Moreover, that agent need not be a liquid or gel that is applied to the adhesive patch just before use of the device. Rather, the anesthetic agent may be incorporated into the absorbent patch during the manufacturing of the device, so that it is ready for use in advance of an ophthalmologicalprocedure. In any case the device may be provided in a sterile package, if desired, so that it may be opened immediately before use. Other changes may be made to the device, e.g., the adhesive need not be in the form of a double-sided tape with or without a removable cover. Thus, the adhesive may be a coating of any suitable material, which may be pre-applied to the portion of the strip that will be brought into contact with the anatomy of the living being below the eye to be treated. If a cover is used on the adhesive patch to protect it, the cover may be of slightly larger size to provide a portion that may be readily grasped by the user to facilitate the peeling or removal of the cover from the adhesive patch when the device is ready for use. It should also be noted that while the device as disclosed above makes use of the adhesive patch on the inner surface of the strip adjacent the proximal end of the strip, it may be used on the outer surface and then folded over in the same direction that the distal portion of the strip which forms the hook-like tab is folded. After it is folded the adhesive patch will be juxtaposed in a confronting position with respect to the portion of the patient’s anatomy to which the device will be releasably secured to hold the device in place during the application of the anesthetic agent to the eye.

Without further elaboration the foregoing will fully illustrate my invention that others may, by applying current or future knowledge, adopt the same for use under various conditions of service.

I claim:

1. A device for numbing an eye of a living being having a globe, a conjunctival sac, a lower palpebral conjunctiva and a bulbar conjunctiva, said device comprising:
a strip of flexible material having a distal end portion, a proximal end portion, and an intermediate portion located between said distal end portion and said proximal end portion;
a foldable line forming an interface between said distal end portion and said intermediate portion;
an absorbent patch located on said distal end portion, said absorbent patch carrying an anesthetic agent, said distal end portion being configured to be folded along said foldable line to form a hook-like tab, said hook-like tab being configured to be introduced into said conjunctival sac between said lower palpebral conjunctiva and said bulbar conjunctiva, whereupon said anesthetic agent engages a portion of said bulbar conjunctiva to numb the eye with said intermediate portion and said proximal end portion of said strip hanging down from the eye and over an external portion of the anatomy of the living being below the eye; and
an adhesive patch located on said proximal end portion, said adhesive patch being configured to be brought into engagement with said external portion of the anatomy of the living below the eye to hold said device in place.

2. The device of claim 1 wherein said strip of flexible material has an outer surface, and an inner surface, wherein said distal end portion terminates at a distal edge and said proximal end portion terminates at a proximal edge, wherein said absorbent patch is located on said outer surface of said distal end portion, and wherein said adhesive patch is located on said inner surface of said proximal end portion adjacent said proximal edge.

3. The device of claim 1 wherein said strip of flexible material has a length in the range of approximately 90 mm to 100 mm between said distal edge and said proximal edge, a width in the range of approximately 10 mm to 15 mm, and a thickness in the range of approximately 0.5 mm to 1 mm.

4. The device of claim 3, wherein said strip of flexible material is approximately 100 mm long, approximately 10 mm wide, and approximately 0.5 mm thick.

5. The device of claim 3 wherein said hook-like tab has a length in the range of 7 mm to 9 mm.

6. The device of claim 5 wherein said hook-like tab has a length of approximately 10 mm.

7. The device of claim 1 wherein said flexible strip is formed of a cardboard or paper.

8. The device of claim 3 wherein said absorbent patch has a thickness in the range of approximately 1 mm to 2 mm.

9. The device of claim 8 wherein said absorbent patch has a thickness of approximately 1.5 mm.

10. The device of claim 1 wherein said absorbent patch comprises gauze or foam.

11. The device of claim 3 wherein said adhesive patch has a length in the range of 7 mm to 9 mm.

12. The device of claim 11 wherein said adhesive patch has a length of approximately 15 mm.

13. The device of claim 1 wherein said adhesive patch is a double-sided tape and said device additionally comprises a removable cover disposed over said adhesive patch to protect said adhesive patch until said device is ready for use, whereupon said removable cover may be grasped to remove it from said adhesive patch to expose said adhesive patch.

14. The device of claim 1 wherein said anesthetic agent comprises lidocaine.

15. A method of numbing an eye of a living being having a globe, a conjunctival sac, a lower palpebral conjunctiva, and a bulbar conjunctiva, said method comprising;
providing a device comprising a strip of flexible material having a distal end portion, a proximal end portion, an intermediate portion located between said distal end portion and said proximal end portion, a foldable line forming an interface between said distal end portion and said intermediate portion, an absorbent patch carrying an anesthetic agent located on said distal end portion, and an adhesive patch located on said proximal end portion;
folding said distal end portion along said foldable line to form a hook-like tab;
introducing said tab into said conjunctival sac between said lower palpebral conjunctiva and said bulbar conjunctiva, whereupon said anesthetic agent engages a portion of said bulbar conjunctiva to numb the eye and said intermediate portion and said proximal end portion of said strip hanging down from the eye and over an external portion of the anatomy of the living being below the eye; and
bringing said adhesive patch into engagement with said external portion of the anatomy of the living below the eye to hold said device in place.

16. The method of claim 15 wherein said absorbent patch of said device is initially provided without said anesthetic agent, and wherein said anesthetic agent is a liquid that is applied to said absorbent patch to be absorbed thereby so that said anesthetic agent is carried by said absorbent patch.

17. The method of claim 15 wherein said adhesive patch is a double-sided tape and said device additionally comprises a removable cover disposed over said adhesive patch to protect said adhesive patch until said device is ready for use, and wherein said method additionally comprises grasped said removable cover to remove it from said adhesive patch to expose said adhesive patch and thereafter bringing said adhesive patch into engagement with said external portion of the anatomy of the living below the eye to hold said device in place.

18. The method of claim 15 additionally comprising leaving said device in place on the living being for a sufficient time for the eye to be numb and once numb cleaning the eye and then preforming any desired procedure thereon.

19. The method of claim 18 wherein said anesthetic agent comprises 4% lidocaine, and wherein said sufficient time is approximately 5 to 7 minutes.

20. The method of claim 19 wherein said procedure comprises injecting the eye.
